(19) United States
(12) Patent Application Publication
(10) Pub. No.: US 2013/0197633 A1
(43) Pub. Date: Aug. 1, 2013

(54) DISPOSITIF DE SUCCION POUR LE TRAITEMENT D UNE PATHOLOGIE OCULAIRE

(71) Applicant: EYE TECH CARE, Rillieux-La-Pape (FR)

(72) Inventors: Fabrizio ROMANO, Beynost (FR); Philippe CHAPUIS, Pommiers (FR); Laurent FARCY, Liergues (FR)

(73) Assignee: EYE TECH CARE, Rillieux-La-Pape (FR)

(21) Appl. No.: 13/707,704

(22) Filed: Dec. 7, 2012

(30) Foreign Application Priority Data
Dec. 8, 2011 (FR) 1161311

(51) Int. Cl. A61F 2/14

(52) U.S. Cl. CPC A61F 2/14
USPC 623/4.1

(57) ABSTRACT

The invention concerns a device allowing the treatment of an ocular pathology, comprising:
a suction ring (1) including a proximal end adapted to abut the eye of a patient and comprising a hollow portion,
an aspiration system (5) connected to the hollow portion to aspirate air in the hollow portion so as to fix the ring onto the patient's eye,
   noteworthy in that the aspiration system is adapted to generate a low vacuum of less than 150 millimetres of mercury, and preferably of substantially 120 millimetres of mercury.

### CROSS REFERENCE TO RELATED APPLICATIONS

This application claims priority to French Application 1161311 filed Dec. 8, 2011.

### BACKGROUND

1. Field of the Invention

The present invention concerns the general technical field of devices for treating an ocular pathology, such as the treatment of glaucoma for example.

2. Description of Related Art

Some surgical procedures on a patient's eye require the use of a suction ring such as described in document EP 1 243 236.

This is notably the case for ocular surgery to treat myopia which entails using instruments comprising a suction ring, a microkeratome instrument and laser.

The method of use of these instruments for the treatment of myopia is the following.

The ring is arranged on the patient's eye and an air vacuum is set up, for example by activating a vacuum pump connected to the suction ring. The aspiration applied by the suction ring causes deformation of the surface of the eye which tends to flatten. When the corneal site is suitably prepared (i.e. the surface of the eye is sufficiently flattened), the practitioner can initiate surgical procedure.

A microkeratome is positioned on the suction ring. The microkeratome is then moved in translation to cut a corneal flap, which is then turned over.

A laser positioned above the region to be treated is activated to excise the exposed fragment of the cornea and obtain the desired correction of vision.

The practitioner then replaces the corneal flap in its initial position.

The use of a suction ring of this type to perform such surgical procedure, on account of the considerable suction force and the positioning of the ring opposite a region, the limbus, not covered by the conjunctiva membrane, causes an increase in the patient's intraocular pressure, this possibly reaching 400 millimetres of mercury or higher.

For some surgical procedures, for example in the treatment of patients suffering from glaucoma, such increase in intraocular pressure is not acceptable.

### SUMMARY

It is one objective of the present invention to propose a suction ring for the treatment of an ocular pathology with which it is possible to limit the increase in intraocular pressure.

For this purpose, the invention proposes a device for treating an ocular pathology, comprising:
a suction ring, in particular conical, including a proximal end arranged to abut the eye of a patient and comprising a hollow portion,
an aspiration system connected to the hollow portion to aspirate air in the hollow portion so as to fix the suction ring on the patient's eye,
   wherein the aspiration system is adapted to generate a low vacuum of between 80 and 150 millimetres of mercury (mmHg), and preferably between 120 and 150 mmHg.

### BRIEF DESCRIPTION OF THE DRAWINGS

FIGS. 1-5 depict embodiments of the present invention.

### DETAILED DESCRIPTION OF A PREFERRED EMBODIMENT

Preferably, the hollow portion is inscribed over a circle whose centre belongs to the axis of symmetry of the ring and which has a diameter of between 14 and 24 millimetres, preferably between 14 and 22 millimetres, and more preferably between 14 and 18 millimetres, so that when the ring is in position on the eye (i.e. the ring is correctly positioned on the eye, or the ring is centred on the eye, or the optical axis of the eye and an axis of symmetry of the ring are coaxial) the hollow portion faces a region of the eye where the sclera is covered by a thin conjunctiva membrane.

The fact that the aspiration zone is placed not in front of the limbus devoid of conjunctiva but at about 2 mm beyond it, i.e. in front of a region where the sclera is covered by a thin conjunctiva membrane, means that it is possible to obtain gentle aspiration of the bulbar conjunctiva, limiting the increase in intraocular pressure of the eye whilst securing the ring onto the eye to be treated.

Preferred but non-limiting aspects of the device of the invention are the following:
the ring comprises an opening from which the cornea of the eye emerges, and a supporting cradle intended to receive ultrasound generating means;
the hollow portion comprises at least one cut-out;
the hollow portion comprises a plurality of cut-outs, each cut-out being connected to the aspiration system whether or not independently;
the hollow portion comprises at least one groove;
the hollow portion comprises a plurality of grooves;
the hollow portion comprises an annular groove.

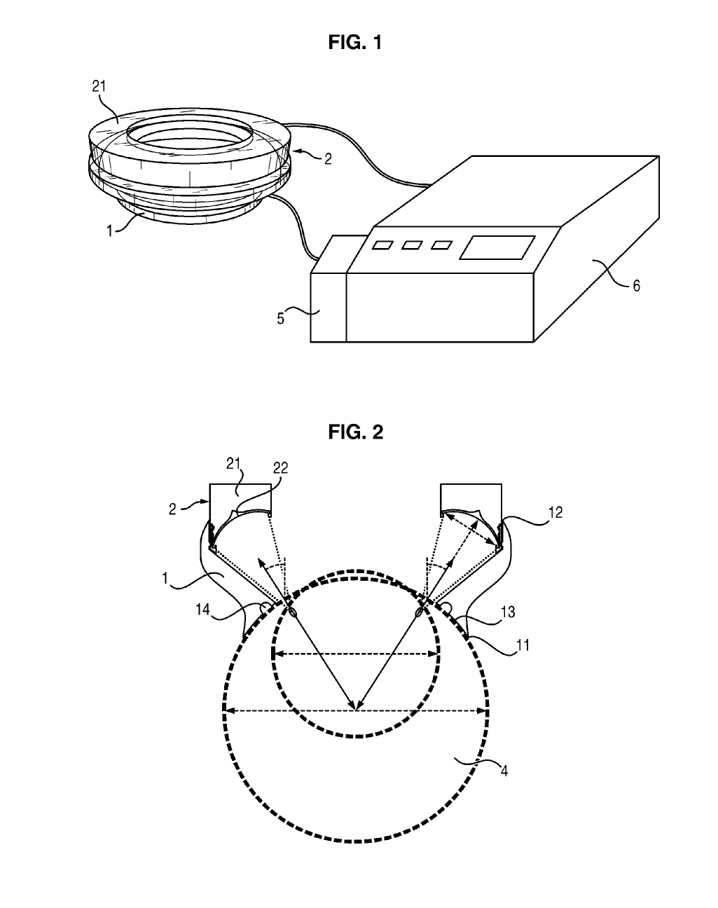

Other advantages and characteristics will become better apparent from the following description of several variants of embodiment given as non-limiting examples in connection with the appended drawings in which FIGS. 1 to 4 illustrate different embodiments of the device according to the invention.

With reference to FIGS. 1 and 2, an illustration is given of one embodiment of the device for treating an ocular pathology.

The device comprises:
a ring 1,
ultrasound generating means 2 to generate ultrasound, and
an aspiration system 5.

The ring 1 is formed of a conical frustum open at the two ends. The small base 11 of the frustum forms the proximal end of the ring 1, and the large base 12 forms the distal end of the ring 1.

The ring 1 allows adequate, constant positioning of the ultrasound generating means 2, both as regards centring and as regards the distance of the ultrasound generating means 2 from the sclera.

The proximal end 11 is intended to come into contact with a patient's eye 4. The distal end 12 of the ring 1 comprises a supporting cradle intended to receive the ultrasound generating means 2.

With reference to FIG. 1, the proximal end 11 of the frustum comprises an outer annular flange 13 able to be applied to the outer surface of the eye 4, at about 2 mm away from the limbus, the limbus being the junction between the cornea and the sclera.

The proximal face of the annular flange 13 has a concave profile, the radius of curvature of the concave profile being substantially equal to the radius of curvature of the eye 4.

The proximal edge 11 of the frustum also comprises a hollow portion 14 connected to the aspiration device 5 by at least one opening passing through the frustum 1 and opening into the hollow portion 14. Advantageously, the aspiration device 5 can be controlled by a control unit 6.

Evidently the aspiration device 5 may be independent without departing from the scope of the invention.

The hollow portion 14 is positioned on the ring so that when in use (i.e. when the ring is positioned on the eye), this hollow portion comes into contact with the conjunctiva membrane. This makes it possible to limit risks of deforming the eye subsequent to the vacuum generated in the hollow portion.

With prior art suction devices, the hollow portion is positioned on the ring so that it comes into direct contact with the surface of the eye, at the limbus. The generation of a vacuum in the hollow portion then induces deformation of the eye (in particular if a high vacuum is generated) tending to increase intraocular pressure.

Conversely, in the present invention, the hollow portion is positioned on the ring so that it comes indirectly into contact with the surface of the eye, the conjunctiva membrane extending between the hollow portion and the surface of the eye.

Therefore, the application of a vacuum in the hollow portion of the device of the invention induces deformation of the conjunctiva membrane. This becomes stretched and slides over the eye. On account of its nonzero coefficient of elasticity, the tensioning of the conjunctiva membrane does not induce sufficient force on the eye to cause deformation thereof. Intraocular pressure in the eye remains substantially invariant, even under the effect of the vacuum applied by the suction device.

Preferably, the position of the hollow portion on the ring is such that it is inscribed over a circle whose centre belongs to the axis of symmetry of the ring and which has a diameter of between 14 and 24 millimetres, preferably between 14 and 22 millimetres, and more preferably between 14 and 18 millimetres. This makes it possible to guarantee that when the ring is positioned on the eye, the hollow portion comes into contact with the conjunctiva membrane.

When applying the vacuum, the conjunctiva membrane becomes deformed. Part of the conjunctiva membrane enters into the hollow portion and tends to block it. Therefore, the holding in position of the suction device is obtained by [symbol] suctioning [symbol] on the conjunctiva membrane whose substantial elasticity allows limiting of the risks of deformation of the eye and hence of an increase in intraocular pressure.

The hollow portion 14 may have different forms.

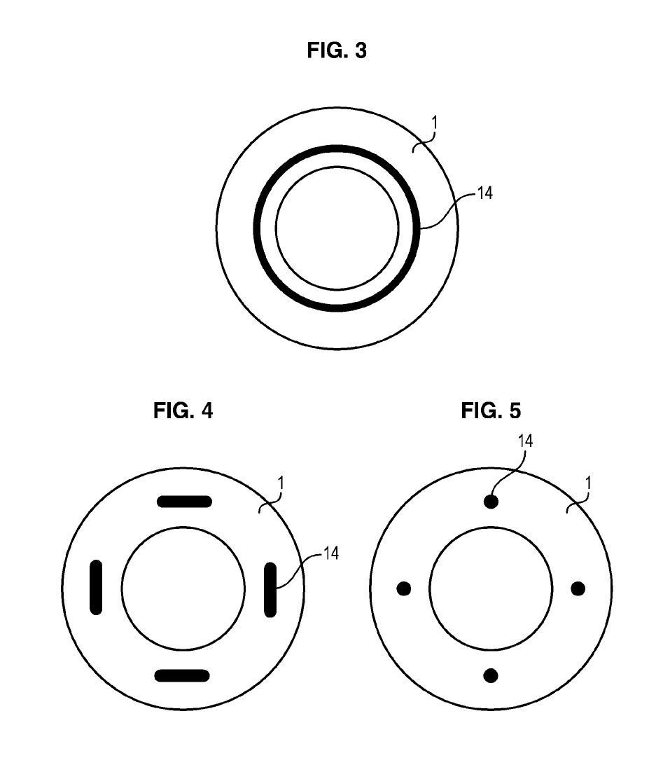

In the embodiment illustrated in FIG. 3, the hollow portion is an annular groove. This makes it possible to ensure suctioning of the ring around the eye over the entire contact surface between the ring and the eye.

In the embodiment illustrated in FIG. 4, the hollow portion consists of a plurality (two or four) of grooves (FIG. 4) and/or cut-outs (FIG. 5). The grooves may be substantially rectilinear and extend along an axis, or they be curved and extend along a portion of a circle to follow the spherical shape of the eyeball.

The fact that the hollow portion consists of a plurality (two or four) grooves and/or cut-outs makes it possible to concentrate the aspirating force on smaller zones whilst limiting risks of deformation of the eye subsequent to aspiration.

Advantageously, each cut-out or groove can be connected to the aspiration system whether or not independently. This allows the ensuring of a uniform vacuum at each of the cut-outs or each of the grooves.

The aspiration system 5 comprises one or more vacuum pumps. When the aspiration system comprises several vacuum pumps, each thereof can be connected to a respective cut-out or respective groove

Advantageously, the aspiration system is adapted to generate a low vacuum of between 80 and 150 millimetres of mercury, preferably 120 millimetres of mercury.

This makes it possible to obtain gentle aspiration of the bulbar conjunctiva, bringing together the ring and the eye to be treated whilst limiting the intraocular pressure of the eye.

The aspiration system may comprise a probe allowing the detection of an ocular parameter corresponding to intraocular pressure, and capable of providing measurements of intraocular pressure. This measurement of intraocular pressure can advantageously be taken into account by control means of the aspiration system to control the vacuum pump(s) so as to maintain the vacuum at a value below 120 millimetres of mercury, and preferably at between 80 and 100 millimetres of mercury.

The operating principle of the aspiration device 5 is the following. The frustum 1 is applied to the patient's eye 4 and the aspiration device 5 is activated. This causes a vacuum to be set up in the annular groove 14 which leads to deformation of the conjunctiva of the eye 4, this deformation forming an O-ring in the annular groove 14.

The frustum 1 is then in tight contact with the eye 4 so that the frustum 1 will follow the movements of the eye 4 throughout treatment procedure. This allows maintaining of the centred position of the apparatus on the axis of vision

After obtaining suctioning of the ring on the eye, the ultrasound generating means—composed of six transducers—can be positioned in the supporting cradle of the ring, and the practitioner can then initiate the desired treatment benefiting from increased safety since the suctioning system via the suction ring allows the position of the probe to be maintained throughout the entire treatment.

Persons skilled in the art will appreciate that numerous modifications can be made to the above-described device without materially departing from the novel teachings presented herein. Therefore, the examples given are evidently only particular illustrations which are in no way limiting.

1. A device for treating an ocular pathology comprising
a suction ring including a proximal end adapted to abut the eye of a patient and comprising a hollow portion,
an aspiration system connected to the hollow portion to aspirate air in the hollow portion so as to fix the ring onto the patient's eye.
wherein the aspiration system is adapted to generate a low vacuum of from 80 to 150 millimetres of mercury.

2. The device according to claim 1 wherein the position of the hollow portion on the ring is such that when the ring is in position on the eye, the hollow portion is in contact with a region of the eye where the sclera is covered with a thin conjunctiva membrane.

3. The device according to claim 1 wherein the hollow portion is positioned on the suction ring so that said hollow portion is inscribed over a circle of a diameter comprised from 14 to 24 millimetres.

4. The device according to claim 1 wherein the aspiration system is adapted to generate a vacuum of substantially 120 millimetres of mercury.

5. The device according to claim 1 wherein the ring comprises an opening from which the cornea of the eye emerges, and a supporting cradle intended to receive ultrasound generating means.

6. The device according to claim 1 wherein the hollow portion comprises at least one cut-out.

7. The device according to claim 1 wherein the hollow portion comprises a plurality of cut-outs.

8. The device according to claim 7 wherein each cut-out is connected independently to the aspiration system.

9. The device according to claim 1 wherein the hollow portion comprises at least one groove.

10. The device according to claim 1 wherein the hollow portion comprises a plurality of grooves.

11. The device according to claim 10 wherein each groove is connected independently to the aspiration system.

12. The device according to claim 1 wherein the hollow portion comprises an annular groove.
